(19) United States
(12) Patent Application Publication
(10) Pub. No.: US 2021/0138088 A1
(43) Pub. Date: May 13, 2021

(54) BISMUTH-IRON OXIDE CONTRAST AGENTS

(71) Applicant: THE TRUSTEES OF THE UNIVERSITY OF PENNSYLVANIA, Philadelphia, PA (US)

(72) Inventors: David Peter Cormode, Philadelphia, PA (US); Pratap Chandra Naha, Philadelphia, PA (US); Andrew Tsourkas, Bryn Mawr, PA (US); Ajlan Al Zaki, Abu Dhabi (AE)

(73) Assignee: THE TRUSTEES OF THE UNIVERSITY OF PENNSYLVANIA, Philadelphia, PA (US)

(21) Appl. No.: 17/116,596

(22) Filed: Dec. 9, 2020

Related U.S. Application Data
(62) Division of application No. 15/052,512, filed on Feb. 24, 2016, now abandoned.
(60) Provisional application No. 62/133,553, filed on Mar. 16, 2015.

(51) Int. Cl. A61K 49/00; A61K 49/18; A61K 49/04

(52) U.S. Cl. CPC A61K 49/0002; A61K 49/0428; A61K 49/1863

(57) ABSTRACT

Nanoparticles for use as a contrast agent, and methods for making and using the nanoparticles, are described, wherein each nanoparticle comprises a core comprising bismuth and iron oxide, and an outer coating (e.g., dextran) surrounding the core. The bismuth-iron oxide nanoparticles can be used in pre-clinical and clinical settings for both computed tomography (CT) and magnetic resonance (MR) imaging.

### CROSS REFERENCE TO RELATED APPLICATIONS

This application is a divisional of, and claims priority to, U.S. patent application Ser. No. 15/052,512 filed Feb. 24, 2016, which claims priority to U.S. Provisional Application No. 62/133,553 filed Mar. 16, 2015, all of which applications are incorporated herein by reference in their entireties.

### STATEMENT REGARDING FEDERALLY SPONSORED RESEARCH

This invention was made with government support under Grant No. NIBIB R00EB012165 awarded by the National Institutes of Health. The government has certain rights in the invention.

### FIELD OF THE INVENTION

The present invention relates to bismuth-iron oxide contrast agents, and methods of using the same.

### BACKGROUND OF THE INVENTION

The field of nanoparticle contrast agents for computed tomography (CT) has grown rapidly over the past decade. Compared to clinically available small molecule contrast agents, nanoparticle-based imaging probes have several advantages, such as the ability to carry a higher payload of contrast-producing material and longer circulation half-lives. Nanoparticles can be efficiently targeted using antibodies, proteins, peptides or other targeting ligands, or used in cell tracking. Also, multiple properties can be integrated into nanoparticles, such as various contrast-generating materials that enable multi-modality imaging or a combination of imaging and therapeutics. Lastly, growing concerns over the biocompatibility of iodinated agents, especially in patients with compromised renal function, has motivated the exploration of new CT contrast agent formulations.

Several research groups have evaluated gold nanoparticles as CT contrast agents. Gold nanoparticles produce strong CT contrast (up to twice that of iodine), are highly biocompatible, can be used for vascular imaging and have been used for targeted CT imaging. An advantage of gold nanoparticles is extensive experience with their synthesis, allowing control over their size, morphology and coating. However, few syntheses of bismuth nanoparticles suitable to be contrast agents have been achieved. Thus, there remains a need for bismuth-based nanoparticles that can work safely and effectively for both CT and magnetic resonance imaging (MRI) purposes.

### SUMMARY OF THE INVENTION

An embodiment of the present invention relates to a nanoparticle for use as a contrast agent, wherein the nanoparticle comprises a core comprising bismuth and iron oxide, and an outer coating surrounding the core. According to preferred embodiments, the outer coating comprises dextran.

Another embodiment of the present invention relates to a contrast agent composition comprising nanoparticles, a carrier, and one or more optional additives, wherein at least some of the nanoparticles (preferably a majority of the nanoparticles, most preferably all of the nanoparticles) each comprises a core comprising bismuth and iron oxide, and an outer coating surrounding the core.

Another embodiment of the present invention relates to a method of using the contrast agent composition comprising administering the contrast agent composition to a subject. Preferably, the method further comprises imaging the subject (e.g., by CT and/or MRI).

Another embodiment of the present invention relates to a method of making nanoparticles comprising co-precipitating iron (preferably ferrous chloride and ferric chloride) and one or more bismuth salts (preferably bismuth nitrate) in the presence of dextran.

### BRIEF DESCRIPTION OF THE DRAWINGS

FIG. 1 illustrates A) an embodiment of a bismuth-iron oxide nanoparticle (BION) reaction scheme; B) transmission electron micrographs of embodiments of dextran-coated BION formulations; C) a plot showing mole % of Bi integrated into embodiments of BION cores after use of different percentages of bismuth nitrate during synthesis; and D) energy dispersive X-ray spectra of embodiments of dextran-coated BION formulations.

FIG. 2 illustrates A) the transverse relaxivity of embodiments of BION formulations; and B) Magnetic hysteresis plots of embodiments of BION formulations.

FIG. 3 illustrates A) a CT image of an embodiment of a BION phantom (iron concentration for the BION formulations is 9.37 mg/ml); B) analysis of the CT attenuation of the BION formulations at various X-ray tube voltages (80 to 140 kV), error bars are standard deviations; C) a magnetic resonance (MR) image of a BION phantom (the iron concentration for the formulations is 0.19 mg/ml).

FIG. 4 illustrates cell viability of A) BJ5ta and B) Hep G2 cells after 24 hours incubation with BION formulations; error bars are standard deviations.

FIG. 5 illustrates in vivo CT imaging of mice injected with an embodiment of BION (Bi-30): A) 3-D volume rendered CT images of a mouse, pre- and 5, 30, 60 and 120 minutes post-injection; B) CT images of a mouse thorax acquired at different time points; the arrow indicates the heart; C) CT images of mouse groin acquired at different time points; the arrow indicates the bladder; D) CT attenuation change of different organs over time, post-BION injection; error bars are standard deviations.

FIG. 6 illustrates A) in vivo MR images of the liver of mice before and 2 hours after injection with an embodiment of BION (Bi-30); B) quantitation of the MR signal intensity in mouse livers compared between pre- and post-injection images; error bars are standard deviations.

FIG. 7 illustrates the biodistribution of an embodiment of BION (Bi-30) in C57BL/6J mice: A) amount of bismuth and iron present in different organs two hours post-injection (values are adjusted for endogenous iron content); B) iron and bismuth content in urine samples collected two hours post-injection; error bars are standard deviations.

FIG. 8 illustrates in vitro biodegradation of an embodiment of BION (Bi-30) in solutions that mimic serum (S) and lysosomal fluid (LF).

FIG. 9 illustrates transmission electron micrographs of embodiments of BION: A) Bi-0 formulation synthesized without ethylene glycol; B) Bi-70; C) Bi-90.

FIG. 10 illustrates saturation of magnetization for different BION formulations.

FIG. 11 illustrates the attenuation of an embodiment of BION as a function of bismuth content and X-ray tube voltage (80-140 kV).

FIG. 12 illustrates whole animal CT images of mice pre- and post-injection with an embodiment of BION (Bi-30) BION (Bi-30 formulation); arrow indicates the bladder.

FIG. 13 illustrates hydrodynamic diameter of an embodiment of BION (Bi-30) after incubation with 10% fetal bovine serum (FBS) at 37° C. for 0, 1 and 24 hours.

FIG. 14 is a schematic illustration of an embodiment of BION and its uses.

### DETAILED DESCRIPTION OF THE INVENTION

Embodiments of the present invention relate to bismuth-iron oxide nanoparticles (BION) that are dextran-coated, wherein the nanoparticles have the advantage of acting as contrast agents for various imaging techniques, including both computed tomography (CT) and magnetic resonance imaging (MRI). Similar to gold, bismuth attenuates X-rays strongly. Additionally, bismuth is inexpensive (about $0.02/g) as compared to gold, and is thought to be a highly biocompatible heavy metal. The dextran-coated BION are preferably biocompatible, biodegradable, possess strong X-ray attenuation properties and can be used as T₂-weighted MR contrast agents.

Embodiments of the invention are described in the article by Naha, et al., Dextran coated bismuth-iron oxide nanohybrid contrast agents for computed tomography and magnetic resonance imaging, Journal of Materials Chemistry B, 2014, 2, pp. 8239-8248, which is incorporated by reference herein, in its entirety and for all purposes.

According to particular embodiments, the invention relates to a nanoparticle, wherein the nanoparticle comprises a core comprising bismuth and iron oxide (e.g., Fe₃O₄), and an outer coating surrounding the core. The outer coating either completely surrounds, or substantially surrounds, the bismuth-iron oxide core. The outer coating may comprise one or more of dextran, carboxydextran, aminated dextran, starch, chitosan, poly(oligo(ethylene glycol) methacrylate-co-methacrylic acid), polyglycidyl methacrylate, poly(vinylalcohol), polyacrylic acid carboxylates, diols, catechols/dopamines, hydroxamic acids, phosphine oxides and silanes. According to preferred embodiments, the outer coating comprises dextran. FIG. 1A and FIG. 14 provide illustrations of an embodiment of a nanoparticle comprising a bismuth-iron oxide core and a dextran coating. According to preferred embodiments, the invention relates to a plurality of the bismuth-iron oxide nanoparticles (BION), wherein the nanoparticles are used as a contrast agent.

According to particular embodiments, the bismuth-iron oxide cores of the nanoparticles comprise between about 1 mole % and about 99 mole % bismuth, or between about 1 mole % and about 80 mole % bismuth, or between about 1 mole % and about 70 mole % bismuth, or between about 5 mole % and about 90 mole % bismuth, or between about 5 mole % and about 80 mole % bismuth, or between about 5 mole % and about 70 mole % bismuth, or between about 5 mole % and about 60 mole % bismuth, or between about 5 mole % and about 50 mole % bismuth, or between about 5 mole % and about 40 mole % bismuth, or between about 5 mole % and about 30 mole % bismuth, or between about 5 mole % and about 25 mole % bismuth. The nanoparticles may be any shape or size, but they preferably have a spherical (or substantially spherical) shape. Regardless of their shape, the nanoparticles preferably have an average hydrodynamic diameter of between about 1 nm and about 500 nm, or between about 30 nm and about 170 nm, or between about 40 nm and about 150 nm, or between about 50 nm and about 150 nm, or between about 75 nm and about 150 nm, or between about 90 nm and about 140 nm.

According to particular embodiments, the dextran coating can be formed from material whose molecular weight is between about 1000 Da and about 2000000 Da, or between about 1500 Da and about 750000 Da, or between about 3500 Da and about 500000 Da, or between about 5000 Da and about 250000 Da, or between about 10000 Da and about 150000 Da, or between about 20000 Da and about 110000 Da. A specific formulation may use dextran of a molecular weight of, for example, about 1000, about 1500, about 3500, about 5000, about 10000, about 20000, about 25000, about 40000, about 60000, about 70000, about 110000, about 150000, about 250000, about 500000, about 750000, about 2000000 Da, or others. The weight % of dextran in the formulation can also vary between about 1% and about 50%, or between about 5% and about 40%, or between about 10% and about 30%, and may take values of, for example, about 5%, about 10%, about 20%, about 30%, about 40% or other values, based on the overall weight of the nanoparticle.

According to particular embodiments, the nanoparticles of the present invention further comprise one or more targeting agents (also known as “targeting ligands”). The targeting agent may be a molecule or a structure that provides targeting of the nanoparticle to a desired organ, tissue or cell. Non-limiting examples of such targeting agents include peptides, antibodies, proteins, nucleic acids, small molecules, etc. The targeting agent(s) are preferably attached to the outer coating for targeted imaging. A nanoparticle comprising one or more targeting agents can be targeted to specific diseased areas of the subject's body.

According to additional embodiments, the invention relates to a contrast agent composition comprising nanoparticles of the present invention, e.g., nanoparticles comprising a bismuth-iron oxide core and an outer coating (e.g., a dextran coating), wherein the nanoparticles are provided in a suitable carrier (preferably a liquid carrier) for administration to a subject. As used herein, the term “carrier” refers to a diluent, adjuvant, excipient, or vehicle with which nanoparticles of the present invention are administered to a subject. Such carriers are preferably liquids; for example, saline, citrate buffer, phosphate-buffered saline, HEPES ((4-(2-hydroxyethyl)-1-piperazineethanesulfonic acid) buffer or Tris buffer are preferred carrier(s). A contrast agent composition of the present invention may also include one or more additives, such as one or more of the following: wetting agents, emulsifying agents, pH buffering agents, antibacterial agents, antioxidants, chelating agents, etc. According to particular embodiments, a method for making a composition of the present invention comprises combining (e.g., mixing or suspending) the nanoparticles with a carrier and one or more optional additives according to known methods.

The terms “subject” and “patient” are used interchangeably herein and refer to a mammalian individual, such as a mouse, rabbit, or human being. In pre-clinical settings, for example, a contrast agent composition of the present invention may be administered to a mouse or rabbit; in clinical settings, the contrast agent composition is preferably administered to a human being.

According to embodiments of the present invention, a method of using bismuth-iron oxide nanoparticles described herein comprises administering the nanoparticles to a subject. According to additional embodiments, a method of using a contrast agent composition described herein comprises administering the contrast agent composition to a subject. For example, the contrast agent composition may be administered to a subject by injection (i.e., intravenously) or by oral administration. The amount of the contrast agent composition that is administered to the subject can be readily determined by one of ordinary skill in the art. For example, the contrast agent composition can be administered to a subject in an amount of between about 1 mg/kg and about 500 mg/kg, or between about 10 mg/kg and about 350 mg/kg, or between about 50 mg/kg and about 200 mg/kg. According to particular embodiments, the method further comprises imaging the subject, preferably by using computed tomography (CT) and/or magnetic resonance imaging (MRI) according to known methods.

According to particular embodiments, a Molday synthesis for iron oxide nanoparticles is employed to make the bismuth-iron oxide nanoparticles, wherein iron (III) and iron (II) chlorides are co-precipitated using ammonia in the presence of dextran. The protocol may be modified by substituting varying amounts of bismuth (III) for iron (III). A described below, a range of such formulations were synthesized to explore the effect of this substitution on the properties of the nanoparticles.

According to particular embodiments, a method of making nanoparticles comprises co-precipitating iron (e.g., ferrous chloride and ferric chloride) and one or more bismuth salts (e.g., bismuth nitrate) in the presence of dextran. Preferably, the method comprises one or more of the following steps: mixing iron (e.g., ferrous chloride and ferric chloride) and one or more bismuth salts (e.g., bismuth nitrate) with a dextran solution to form a dextran-iron-bismuth solution; adding ammonium hydroxide (e.g., 28-30% or other concentrations) to the dextran-iron-bismuth solution; heating the resulting solution to form a nanoparticle suspension; centrifuging the suspension; and collecting the resulting nanoparticles from the suspension. Preferably, the iron (e.g., ferrous chloride and ferric chloride) is dissolved in water and the bismuth salt(s) (e.g., bismuth nitrate) are dissolved in a polyol, e.g., ethylene glycol, prior to addition to the dextran solution. According to particular embodiments, the method of making nanoparticles further comprises attaching one or more targeting agents onto the coating according to known methods (e.g., one or more targeting agents selected from the group consisting of peptides, antibodies, proteins, nucleic acids, small molecules, and a combination thereof).

For example, the method may comprise one or more of the following steps: dissolving dextran in water to form a dextran solution; removing oxygen from the solution (e.g., by purging the solution with nitrogen gas while stirring); mixing iron (e.g., ferrous chloride and ferric chloride) and one or more bismuth salts (e.g., bismuth nitrate) with the dextran solution (preferably, the iron is dissolved in water and the bismuth salt is dissolved in a polyol, e.g., ethylene glycol, prior to addition to the dextran solution) to form a dextran-iron-bismuth solution; adding ammonium hydroxide (e.g., 28-30%) to the dextran-iron-bismuth solution; heating the resulting solution (e.g., at about 90° C. for about an hour) to form a nanoparticle suspension; stirring the suspension at room temperature (e.g., overnight); centrifuging the suspension (e.g., at 20 k rcf for 30 minutes); collecting the supernatant and concentrating it (e.g., using ultrafiltration tubes) to obtain the nanoparticles; and washing the nanoparticles (e.g., with citrate buffer using 100 kDa MW diafiltration columns).

As shown in Table 1, a range of bismuth-iron oxide nanoparticle (BION) formulations can be synthesized, for example, by substituting different percentages of iron (e.g., ferric chloride) with a bismuth salt (e.g., bismuth (III) nitrate). According to particular embodiments, the amount of bismuth salt (e.g., bismuth nitrate) mixed with the dextran solution (relative to the total amount of bismuth salt, ferrous chloride and ferric chloride) is between about 1 wt % and about 99 wt %, or between about 10 wt % and about 90 wt %, or between about 25 wt % and about 90 wt %, or between about 25 wt % and about 75 wt %, or between about 25 wt % and about 60 wt %, as shown in Table 1. According to particular embodiments, the amount of ferrous chloride plus ferric chloride mixed with the dextran solution (relative to the total amount of bismuth salt, ferrous chloride and ferric chloride) is between about 10 wt % and about 90 wt %, or between about 10 wt % and about 75 wt %, or between about 25 wt % and about 75 wt %, or between about 40 wt % and about 75 wt %, as shown in Table 1. According to preferred embodiments, the amount of bismuth salt (e.g., bismuth nitrate) mixed with the dextran solution (relative to the total amount of bismuth salt, ferrous chloride and ferric chloride) is between about 25 wt % and about 60 wt % (e.g., as shown in the Bi-30 and Bi-50 formulations described below).

According to particular embodiments, it was found that inclusion of bismuth resulted in marked increases in X-ray attenuation, in proportion to the amount of bismuth in the formulation (see, e.g., FIG. 11). According to preferred embodiments, the nanoparticles of the present invention produce no cytotoxic effects. The in vivo experiments described below indicate that nanoparticles of the present invention are preferably effective in pre-clinical and clinical settings as a dual modality contrast agents for CT and MR imaging.

The embodiments of the invention are described above using the term “comprising” and variations thereof. However, it is the intent of the inventors that the term “comprising” may be substituted in any of the embodiments described herein with “consisting of” and “consisting essentially of” without departing from the scope of the invention. Unless specified otherwise, all values provided herein include up to and including the starting points and end points given.

The following examples further illustrate embodiments of the invention and are to be construed as illustrative and not in limitation thereof.

### EXAMPLES

Provided below are the results of synthesis and characterization of several dextran-coated bismuth iron oxide nanoparticle (BION) formulations and in vivo imaging experiments using a selected formulation (Bi-30). BION were synthesized through co-precipitation of ferrous chloride, ferric chloride and bismuth nitrate in the presence of dextran. A range of BION formulations were synthesized by substituting several different percentages of ferric chloride with bismuth (III) nitrate. Each BION formulation was characterized using techniques such as transmission electron microscopy (TEM), dynamic light scattering (DLS), inductively coupled plasma optical emission spectroscopy (ICP-OES), relaxometry, energy dispersive X-ray spectroscopy (EDS) and magnetic hysteresis measurements. The biocompatibility of the nanoparticles was evaluated via in vitro incubation with hepatocytes (Hep G2) and fibroblasts (BJ5ta). Taken together, the results of these experiments indicated that Bi-30 was the preferred formulation to test in vivo. CT and MR imaging experiments, biodistribution measurements and biodegradation studies were carried out using this formulation in mice, which demonstrated the potential of this agent as a dual CT/MRI contrast agent and that it is degradable and excretable in vivo.

### Synthesis and Purification of BION.

Several different BION formulations were synthesized by co-precipitation of iron and bismuth salts in the presence of dextran using a modified version of a protocol previously reported for the synthesis of dextran coated iron oxide. In this process, 12.5 g of dextran T-10 (Pharmacosmos, Holbaek, Denmark) were dissolved in 25 ml of deionized (DI) water. The resulting solution was placed in an ice bath and was purged with nitrogen gas for 30 minutes while stirred, to completely remove oxygen from the flask. For each formulation, varying amounts of ferric chloride and bismuth nitrate, but a fixed amount of ferrous chloride (all purchased from Sigma-Aldrich, St. Louis, USA), as summarized in Table 1, were added to the dextran solution. As the amount of bismuth nitrate was increased, the amount of ferric chloride used in the reaction was reduced, to maintain the molar ratios between the Fe³⁺/Bi³⁺ and Fe²⁺ ions. The ferric chloride and ferrous chloride were each dissolved in 6.5 ml of DI water whereas the bismuth nitrate was dissolved in 3.5 ml of ethylene glycol prior to addition to the dextran solution (ethylene glycol was also added to the reaction when no bismuth was used). Notably, in the absence of bismuth incorporation, there was no difference found in particle size or transverse (T₂) relaxivity on the presence or absence of ethylene glycol. A transmission electron microscopy (TEM) image for Bi-0 formulation synthesized without ethylene glycol is presented in FIG. 9A.

Fifteen ml of concentrated ammonium hydroxide (28-30%) were added to the dextran-iron-bismuth solution using a syringe pump. The ammonium hydroxide flow rate was set to 0.3 μl/min for the first 2.5 hours and then increased to 0.6, 0.9 and 1.2 μl/min for 1 hour each, consecutively; the remainder of the ammonium hydroxide was added to the dextran solution at a rate of 4 μl/min. After addition of ammonium hydroxide, the nanoparticle suspension was heated to 90° C. for an hour and then stirred at room temperature overnight. The resulting nanoparticle suspension was centrifuged at 20 k rcf for 30 minutes. The supernatant was collected and concentrated to 15 ml using ultrafiltration tubes (molecular weight cut off 100 kDa, Sartorius Stedim Biotech, Germany). The nanoparticles were washed with citrate buffer (0.15 M sodium chloride and 20 mM sodium citrate dehydrate, pH 7.4) using 100 kDa MW diafiltration columns (SPECTRUM, Calif., USA) continuously for 16 h using a peristaltic pump. After purification, the resulting BION formulations were stored at 4° C. Several formulations were synthesized and termed as Bi-0 Bi-10, Bi-30, etc., where the number indicates the percentage of ferric chloride replaced with bismuth nitrate, as summarized in Table 1.

TABLE 1
Amounts of the reagents used in the synthesis of BION.

| Formulation | FeCl₃•6H₂O (g) | FeCl₂•4H₂O (g) | Bi(NO₃)₃•5H₂O (g) |
|---|---|---|---|
| Bi-0 | 0.98 | 0.36 | 0 |
| Bi-10 | 0.88 | 0.36 | 0.17 |
| Bi-30 | 0.68 | 0.36 | 0.53 |
| Bi-50 | 0.49 | 0.36 | 0.88 |
| Bi-70 | 0.29 | 0.36 | 1.23 |
| Bi-90 | 0.09 | 0.36 | 1.59 |

### Characterization

### Dynamic Light Scattering and Zeta Potential.

The hydrodynamic diameter and zeta potential of each BION formulation were measured using a Nano-ZS 90 (Malvern Instrument, Malvern, UK). Hydrodynamic diameter and zeta potential measurements were performed at 25° C. using 1.5 and 1 ml of diluted BION (10 μl from stock was diluted with 2 ml of freshly filtered (0.2 μm) DI water).

### Transmission Electron Microscopy.

Transmission electron microscopy of each BION formulation was performed using a JEOL 1010 microscope operating at 80 kV. Ten μl of diluted BION (10 μl from stock was diluted with 1 ml of DI water) was dropped onto carbon coated copper grid (FCF-200-Cu, Electron Microscopy Sciences, Hatfield, Pa., USA), and allowed to evaporate before imaging.

### Energy Dispersive X-Ray Spectroscopy.

Elemental analysis of each BION formulation was performed using energy dispersive X-ray spectroscopy. TEM grids were prepared as described above and the elemental analysis was performed using a JEOL 2010F microscope operating at 80 to 200 kV.

### Inductively Coupled Plasma Optical Emission Spectroscopy.

The amount of iron and bismuth present in each BION was determined using inductively coupled plasma optical emission spectroscopy (ICP-OES, Spectro Genesis ICP). In brief, 5, 10 and 25 μl of BION from the stock of each formulation were dissolved in 1 ml of concentrated nitric acid (70%). The final volume of each sample was adjusted to 10 ml using deionized water. Bismuth and iron analytical standards were purchased from Fisher Scientific (Pittsburgh, USA). The concentrations of bismuth and iron were determined for each sample, multiplied by the dilution factor and the concentrations thus obtained were averaged to obtain the final bismuth and iron concentration for each BION formulation.

### T2 Relaxivity Measurement.

The T2 relaxivity of each BION formulation was measured using a tabletop Minispec MR (Bruker mq60 MR) relaxometer operating at 1.41 T (60 MHz). T2 values were measured for BION formulation whose iron concentration ranged from 0.1 to 0.6 mM. The transverse relaxivity was calculated from the slope of 1/T2 plotted against iron concentration for each formulation.

### Magnetic Properties.

The magnetic properties of each BION formulation were determined using an alternating gradient magnetometer (Princeton Instruments Corporation, Princeton, N.J., USA). In brief, 5 μl of BION suspensions (12 mg Fe/mil) were dropped on to 4×4 mm² cover glass slides and allowed to dry. The magnetic properties of each BION formulation were estimated from their hysteresis curves.

### Phantom Construction and Imaging

### BION Phantom CT Imaging.

The CT phantom was constructed similarly to a described previously protocol. In brief, the concentration of each BION formulation was adjusted to 9.37 mg Fe/ml, whereas the bismuth concentration varied in each formulation (the concentrations of bismuth in Bi-0, Bi-10, Bi-30 and Bi-50 were 0, 1.98, 6.34 and 6.38 mg/ml respectively). These samples were prepared in triplicate in 1.5 ml centrifuge tubes. The tubes were placed in a rack, which was covered in parafilm and the rack placed in a plastic container (24 cm in width) that was filled with water up to 21 cm in height, to simulate the attenuation effects of the abdomen of a patient. The phantom was scanned using a Siemens Definition DS 64-slice clinical CT scanner at 80 kV (550 mA), 100 kV (440 mA), 120 kV (352 mA) and 140 kV (308 mA) with a matrix size of 512×512, field of view 37×37 cm, reconstruction kernel B30f and slice thickness of 0.6 cm. Images were analyzed using Osirix 64 bit (v3.7.1). The attenuation value, in Hounsfield Units (HU) for each sample tube was determined from three different slices and averaged for each sample.

### BION Phantom MR Imaging.

BION samples at a concentration of 0.195 mg Fe/ml were prepared in triplicate in 1.5 ml centrifuge tubes. The tubes were placed in a rack, which was then placed in a hot solution of 2% agarose and 0.35 mM manganese chloride solution. The phantom was cooled to 4° C. to allow the agarose to solidify. The BION phantom was scanned using a 3T MRI system (Tim Trio Model, Siemens Healthcare, Erlangen, Germany). We computed T2 using a spin echo pulse sequence. The scanning parameters used were: echo time (TE)=5.8 ms, repetition time (TR)=10 sec, slice thickness=3 mm, flip angle (FA)=180 degrees, acquisition matrix 184×256, field of view (FOV) 165×230. The MR images of BION phantom were analyzed using Osirix v.3.0.1 32-bit.

### In Vitro Cell Viability Assay

### Cell Culture.

Hep G2 (human hepatocellular liver carcinoma) and BJ5ta (human foreskin fibroblast) cells were purchased from ATCC (Manassas, Va., USA). The Hep G2 cells were maintained in Eagle's Minimum Essential Medium, 10% fetal bovine serum (Gibco, Grand Island, N.Y. USA), 45 IU/ml penicillin and 45 IU/ml streptomycin (Gibco). The BJ5ta cells were maintained in a culture medium containing 4 parts of Dulbecco's Modified Eagle's Medium (ATCC), 1 parts of medium 199 (ATCC), 10% fetal bovine serum (Gibco) and 0.01 mg/ml of hygromycin B (Sigma-Aldrich). The cells were grown at 37° C. in 5% CO₂ humidified incubator.

### Cytotoxicity Assay.

In vitro cytotoxicity of each BION formulation was examined in Hep G2 and BJ5ta cells using the MTS ((3-(4,5-dimethylthiazol-2-yl)-5-(3-carboxymethoxyphenyl)-2-(4-sulfophenyl)-2H-tetrazolium) assay (CellTiter 96 cell proliferation assay kit; Promega, Madison, Wis., USA). The assay was performed according to the manufacturer instructions and as reported elsewhere.

In brief, the assay was performed in a 96 well flat bottom micro plate with 10,000 cells seeded in each well. Three different concentrations (i.e., 10, 50 and 100 μg Fe/mil) of each BION formulation were tested in both cell lines. Six replicate wells were used for each control and test concentration per microplate. After 24 hours incubation with BION, the cell monolayer was rinsed gently with sterile PBS. 20 μl of MTS/phenazine methosulfate (PMS) solution and 100 μl of cell culture medium were added to each well and the plates subsequently incubated at 37° C. for 1 hour. After this incubation, the absorbance was measured at 490 nm using a micro plate reader. Three independent experiments were performed for each exposure dose and each BION formulation. The percentage of relative cell viability was calculated and the data presented as mean±standard deviation (n=3).

### In Vivo Characterization of BION

### Animal Experiments.

All animal experiments were performed using wild-type C57BL/6J inbred mice (Jackson Laboratory, Bar Harbor, Me., USA). All in vivo experiments were performed with Bi-30 at a dose of 350 mg Bi/kg body weight, administered via the tail vein.

### In Vivo CT Imaging.

For CT imaging experiments, pre and post-injection CT images of mice was acquired using a MicroCAT II (Imtek, Inc, Knoxville, Tenn., USA). 8 week old wild-type C57BL/6J mice (n=3) were anesthetized via isoflurane prior to a pre-injection CT scan and then injected with BION (Bi-30). After this injection, mice were scanned at different time intervals post-injection i.e. 5, 30, 60 and 120 minutes. CT images were acquired using the following parameters: slice thickness 100 μm, field of view 51.2 mm×76.8 mm, tube voltage 80 kV and tube current was 500 μA. The reconstruction kernel used a Feldkamp cone beam correction and Shepp-Logan filter. The CT images were analyzed using Osirix 64 bit (v3.7.1). The attenuation values in Hounsfield Units (HU) for several organs (heart, liver, spleen and bladder) were recorded from three different slices and averaged. The data presented is the relative attenuation compared to pre-injection scan (mean±SD).

### In Vivo MR Imaging.

For MR imaging experiments, pre and post-injection MR images of mice were acquired using a 9.4 T magnet interfaced to a Varian INOVA console (Varian Medical Systems, Palo Alto, Calif., USA). In brief, 8-week-old wild-type C57BL/6J mice (n=3) were anesthetized with isoflurane prior to the pre-injection MR scan. The mice were scanned again two hours after injection with BION (Bi-30). MR images were acquired using the following parameters: TR=200 ms, TE=5 ms, flip angle 20°, number of acquisitions 8 and slice thickness 0.5 mm. The images were analyzed using ImageJ. For each mouse, three different axial slices were chosen for analysis from both the pre and post-injection scans. The signal to noise ratio (SNR) of the liver and adjacent muscle in each slice was calculated by dividing the standard deviation of the background noise. The data is presented as the mean±SD.

### Biodistribution.

The biodistribution of BION (Bi-30) was investigated in 8-week-old wild-type C57BL/6J mice (n=4). A control group of mice (n=4) was employed for estimation of iron content in different organs. At 2 hours post injection of Bi-30, the mice were sacrificed and perfused via the left ventricle with 20 ml PBS. After perfusion, the liver, lungs, heart, spleen and kidneys were harvested. These organs were also harvested in the same way as described above for the control group. All the organs were weighed, diced into small pieces and digested in 800 μl of concentrated nitric acid at 75° C. for nearly 16 hours. Urine samples were collected from the treated (after 2 hour post injection) and control group. The bismuth and iron content were determined using the ICP-OES. Data are presented as mean±SD (n=4).

### In Vitro Biodegradation Experiments.

Samples of the Bi-30 formulation (4 mg Fe) were suspended in either 1 ml of 10% fetal bovine serum (FBS) (in PBS, pH 7.4) or 20 mM citrate buffer (pH 5.5) in 1.5 ml centrifuge tubes and incubated at 37° C. in an oven. At different time intervals (such as 1, 2, 4, 6, 24, 48, 72, 96, 120, 144 and 160 hours) the supernatant from each sample was collected (after centrifugation at 20800 rcf for 20 min) and nanoparticle pellets were resuspended with fresh buffer. Then the tubes were incubated in the oven for next round of sample collection. The released amount of bismuth and iron were analyzed in the samples using ICP-OES.

### Results and Discussion

### Synthesis and Characterization of BION Formulations.

Dextran coated BION formulations were synthesized by co-precipitation of Fe(III), Fe(II) and Bi(III) in the presence of dextran via the addition of ammonium hydroxide (FIG. 1A). A range of formulations (Bi-0, Bi-10, Bi-30, Bi-50, Bi-70 and Bi-90) were synthesized by use of different amounts of bismuth nitrate. The ICP-OES results showed that the formulations with higher percentages of bismuth nitrate used during synthesis (i.e. Bi-70 and Bi-90) exhibited a very low recovery yield (Table 2). These formulations were not pursued after performing preliminary characterization.

The hydrodynamic diameter of the BION formulations increased with increasing amounts of bismuth used in the synthesis (Table 2), even though the cores formed did not increase in size, as evidenced by the TEM images (FIG. 1B). ICP-OES revealed that the more bismuth nitrate was used in the reaction, the more bismuth was integrated into the nanoparticles, generally speaking (Table 2). FIG. 1C shows the mole % of bismuth present in each BION formulation versus the mole % of bismuth nitrate incorporated during synthesis of each BION formulation. When 10 or 30% bismuth nitrate was added to the reaction, a slightly lower percentage of bismuth was found in the final formulations. However, when 50% bismuth nitrate was used, the bismuth content in the product was only 23% and the reaction yield decreased. To further probe the inclusion of bismuth into iron oxide nanoparticles, EDS was performed (FIG. 1D). When bismuth was added to the reaction mixture, bismuth peaks clearly arose in the spectra (the copper peaks are signal from the grid material). The results suggested that bismuth was integrated into the iron oxide cores in each case. The zeta potential of the different BION formulations did not change substantially with the integration of bismuth into the iron oxide core (Table 2).

TABLE 2
Characterization data of the dextran coated BION formulations.

| | Formulation | % yield | % yield | Hydrodynamic | Zeta | T₂ relaxivity |
|---|---|---|---|---|---|---|
| Formulations | details | (iron) | (bismuth) | diameter (nm) | potential (mV) | (mM⁻¹s⁻¹) |
| Bi-0 | Iron 100% | 16.8 | N/A | 35.6 ± 0.6 | −11.7 ± 0.9 | 74 |
| Bi-10 | Iron-90% | 18.2 | 28.2 | 56.5 ± 0.4 | −8.4 ± 0.5 | 1.05 |
| | Bismuth-10% | | | | | |
| Bi-30 | Iron-70% | 12.5 | 16.6 | 98 ± 0.8 | −8.5 ± 1.9 | 0.4 |
| | Bismuth-30% | | | | | |
| Bi-50 | Iron-50% | 10.3 | 6.8 | 123 ± 0.9 | −8.7 ± 1.2 | 0.22 |
| | Bismuth-50% | | | | | |
| Bi-70 | Iron-30% | 3.7 | 1.7 | 151 ± 0.8 | −7.88 ± 0.3 | 0.20 |
| | Bismuth-70% | | | | | |
| Bi-90 | Iron-10% | 0.7 | 0.5 | 178 ± 2.4 | −14.7 ± 0.4 | — |
| | Bismuth-90% | | | | | |

The relationship between T2 relaxivity and the amount of bismuth used in synthesis is shown in FIG. 2A, while the magnetic hysteresis plots for the BION formulations are presented in FIG. 2B. The T2 relaxivities decreased with increased bismuth content in the nanoparticle. The magnetization of the BION formulations also decreased with increased bismuth content in the nanoparticle formulation (FIG. 10). As the addition of bismuth disrupts the magnetic properties of the nanoparticles, it is likely that bismuth is integrated into the iron oxide crystal lattice. The data from the various characterization techniques (ICP-OES, EDS, T2 relaxivity and magnetization) strongly suggests that bismuth was integrated into the iron oxide core, as opposed to the reaction creating a mixture of bismuth and iron oxide nanoparticles.

BION Phantom Imaging with CT and MRI.

CT images, acquired at 140 kV, of a phantom containing several BION formulations at the same iron concentration (9.4 mg/ml) is presented in FIG. 3A. The X-ray attenuation was quantified for each BION formulation at several X-ray tube voltages (FIG. 3B). This data shows that iron oxide alone (Bi-0) attenuates X-rays poorly, with only ˜50 HU produced by this concentration, depending on the X-ray voltage used (CT attenuation scales approximately with Z³, so iron is not expected to strongly attenuate X-rays). Inclusion of bismuth resulted in marked increases in the X-ray attenuation, in proportion to the amount of bismuth in the formulation (FIG. 11). Attenuation was strongest at 80 kV, giving Hounsfield unit values about 20% higher than for the other X-ray tube voltages. The attenuation did not change significantly between 100 and 140 kV. This pattern of unchanging attenuation with changing X-ray tube voltage is similar to results we have previously reported for bismuth nanoparticles. This is likely related to the high k-edge of bismuth (90.8 keV) compared to the average energy of the X-ray beam for the specific CT scanner used (56 keV for 80 kV scan to 76 keV for 140 kV scan).

A magnetic resonance image of a phantom containing different BION formulations is shown in FIG. 3C. The Bi-0 formulation produced the strongest contrast, while the contrast produced by Bi-10, Bi-30 and Bi-50 was lower. Nevertheless, the data predicted that BION formulations should still be effective as MR contrast agents given that the doses required for CT are in the range of hundreds of mg/kg. For a BION formulation, hundreds of mg/kg of both Bi and Fe would be used. So despite low MR contrast-inducing properties, due to the large doses that would be used, we expected to see significant MR contrast produced in vivo.

### Cell Viability.

The results of cell viability measurements for BION formulations incubated with BJ5ta or Hep G2 cells are presented in FIG. 4. No cytotoxic effect was observed in either cell line after 24 hours of incubation with the BION formulations at the doses used, indicating that the agents were biocompatible. The characteristics of the BION formulations in terms of biocompatibility, relaxivity, and surface charge were very similar. However, Bi-30 and Bi-50 had the highest bismuth inclusion, maximizing the CT contrast properties of the nanoparticles, but the yield of B-30 was higher, therefore we selected this formulation for in vivo experiments.

### In Vivo Experiments

### In Vivo CT Imaging

We studied the CT contrast generation of Bi-30 in wild type mice using a microCT scanner operating at 80 kV. Mice were injected via the tail vein at a dose of 350 mg Bi/kg, similar to the doses of iodinated CT contrast agents used in patients. Representative pre- and post-injection CT images are presented in FIG. 5. Strong X-ray attenuation in the blood vessels and heart of the mice was observed (FIGS. 5A and B). CT contrast in the heart and the blood vessels decreased over time, whereas CT attenuation in the bladder increased from 5 minutes to 30 minutes post-injection and remained constant thereafter (FIG. 5C, FIG. 12). The contrast produced in the bladder indicated that some bismuth was being excreted in urine, which was surprising, as the nanoparticles are too large to be excreted renally in their intact form. Potentially this excretion could be due to degradation of the nanoparticles, a hypothesis that we tested in subsequent sections. CT attenuation in the heart and blood vessel was observed for the duration of our measurements, i.e. until 120 minutes post-injection (FIG. 5A), which indicated that BION were in the systemic circulation. Presumably contrast would also be observed over longer timeframes, if measured. The X-ray attenuation in different organs (such as heart, liver, spleen and bladder) was quantified and compared to that in the pre injection scans (FIG. 5D). The results for the heart and bladder confirm the visual impressions from FIG. 5A-C. Some contrast in the liver and spleen was observed, indicating nanoparticle accumulation in these organs, as is typically observed.

### In Vivo MR Imaging

MR images of a mouse liver before and after Bi-30 injection are presented in FIG. 6A. While whole body CT imaging can be easily performed using preclinical scanners, MRI using small bore scanners can only image limited anatomical regions. As we observed significant nanoparticle accumulation in the liver via CT, we therefore selected that organ for imaging with MRI. As can be seen, a significant loss of MR signal in the liver (at 2 hours post-injection) was observed compared to the pre-injection image. Even though the T2 relaxivity of Bi-30 formulation is low, at only 0.4 mM⁻¹ s⁻¹, contrast in MRI is due to the product of relaxivity and concentration. Thus the high dose used was sufficient to generate MR contrast in vivo. These in vivo CT and MR imaging experiments indicated that the BION (Bi-30) is effective as a dual modality contrast agent.

### Biodistribution.

Our observations of CT contrast in the bladder motivated investigation of biodistribution at 2 hours post-BION injection, to determine whether bismuth and iron were being excreted via this route. This data is presented in FIG. 7; we found that 11% ID/g accumulated in the spleen, while in rest of the organs (heart, lungs, kidneys and liver) ≤6% ID/g was found. The bismuth content of the kidney was found to be significantly higher than that of iron, whereas there was no significant difference in the other organs. Furthermore, we found that bismuth was excreted in urine in significantly higher quantities than iron (FIG. 7B). This suggested that the BION degraded in vivo and that the released ions were excreted in urine. The greater content of bismuth compared to iron in the kidneys and urine may be due to sequestration of released iron by ferritin and other iron sinks within the body, whereas no such system exists for bismuth and therefore bismuth ions are free to be filtered in the kidneys. It has been reported that dextran coated iron oxide nanoparticles can be internalized by cells via endocytosis, degraded in the acidic environment and iron released into the cytoplasm. The iron may be used by the cells or stored in ferritin and eventually participate in the synthesis of red blood cells.

### Biodegradation.

To probe the biodegradation of Bi-30, we incubated samples in 10% FBS at 37° C. After 0, 1 and 24 hours of incubation, the hydrodynamic diameter was measured. The hydrodynamic diameter decreased over time from 98 to 90 nm after one hour and to 48 nm after 24 hours (FIG. 13). This result supported the hypothesis of BION degradation under biological conditions. Additionally, we investigated the release of bismuth and iron by incubating Bi-30 formulation for 7 days in two different conditions, i.e. 10% FBS (mimicking the blood) and a citrate based lysosomal mimicking fluid. The nanoparticles are injected into the blood and many of them are likely taken up into endosomes, therefore these are relevant conditions to test. In the lysosomal mimicking fluid, we observed that 80% of Bi and Fe was released within 4 hours and 89% at 7 days. However, when incubated in 10% serum, only 19% and 29% of Bi and Fe was released at 4 hours and 7 days, respectively (FIG. 8). These results suggest that BION may be degraded in vivo, causing bismuth and iron ions to be released.

In this study, the characterization results suggested that the bismuth is integrated into the iron oxide core. Strong X-ray attenuation was observed from the composite nanohybrid. The magnetic properties and transverse relaxation of the BION formulations were decreased due to the presence of bismuth in the nanoparticle core. In vitro cell viability experiments demonstrated the biocompatibility of the BION. In vivo CT imaging revealed strong X-ray attenuation in the heart and blood vessels over a sustained period. We observed strong contrast in mouse liver post-injection during in vivo MR imaging experiments. The biodistribution and in vitro degradation experiments suggests that the nanoparticles degrade over time and excreted in urine. In summary, dextran coated BION are biocompatible, biodegradable, possess strong X-ray attenuation at CT and also perform as T2-weighted MR contrast agents. Therefore, BION can be used as a dual imaging probe for both CT and MRI.

The following references are incorporated by reference herein, in their entireties and for all purposes:

What is claimed is:

1. A method of using a contrast agent composition comprising nanoparticles, a carrier, and one or more optional additives, wherein at least some of the nanoparticles comprise:
a core comprising bismuth and iron oxide; and
an outer coating surrounding the core;
the method comprising:
administering the contrast agent composition to a subject.

2. The method of claim 1, wherein the contrast agent composition is administered to the subject in an amount of between about 1 mg/kg and about 500 mg/kg.

3. The method of claim 1 further comprising:
imaging the subject using one or more techniques selected from the group consisting of: computed tomography (CT) and/or magnetic resonance imaging (MRI).

4. A method of making nanoparticles for use as a contrast agent, the method comprising:
co-precipitating iron and one or more bismuth salts in the presence of dextran and ammonia.

5. The method of claim 4, wherein the iron comprises ferrous chloride and ferric chloride, and the one or more bismuth salts comprise bismuth nitrate.

6. The method of claim 5, wherein the amount of the bismuth nitrate, relative to the total amount of the bismuth nitrate, the ferrous chloride and the ferric chloride, is between about 1 wt % and about 99 wt %.

7. The method of claim 5 further comprising:
mixing a dextran solution with the ferrous chloride, the ferric chloride and the bismuth nitrate.

8. The method of claim 5, further comprising:
dissolving the ferrous chloride and the ferric chloride in water;
dissolving the bismuth nitrate in a polyol;
dissolving the dextran in water to form the dextran solution; and
mixing the dextran solution with the ferrous chloride, the ferric chloride and the bismuth nitrate.

9. The method of claim 4 comprising:
mixing the iron and the one or more bismuth salts with a dextran solution to form a dextran-iron-bismuth solution; and
adding ammonium hydroxide to the dextran-iron-bismuth solution.

10. The method of claim 9 further comprising:
heating the dextran-iron-bismuth solution to form a nanoparticle suspension;
centrifuging the nanoparticle suspension; and
collecting nanoparticles from the nanoparticle suspension.

11. The method of claim 4 further comprising:
mixing the nanoparticles with a carrier and one or more optional additives to form a contrast agent composition.

12. The method of claim 7, wherein the amount of ferrous chloride plus ferric chloride mixed with the dextran solution, relative to the total amount of the bismuth nitrate, ferrous chloride, and ferric chloride, is between about 10 wt % and about 90 wt %.

13. The method of claim 7, wherein the amount of bismuth nitrate relative to the total amount of the bismuth nitrate, the ferrous chloride, and the ferric chloride, is between about 25 wt % and about 60 wt %.
